(19) United States
(12) Patent Application Publication
(10) Pub. No.: US 2013/0331654 A1
(43) Pub. Date: Dec. 12, 2013

(54) Transmission-Efficient Light Couplings and Tools and Systems Utilizing Such Couplings

(71) Applicant: The Nathaniel Group, Inc., Vergennes, VT (US)

(72) Inventor: James Hermanowski, Waterbury, VT (US)

(21) Appl. No.: 13/915,368

(22) Filed: Jun. 11, 2013

Related U.S. Application Data
(60) Provisional application No. 61/658,350, filed on Jun. 11, 2012.

(51) Int. Cl. A61B 1/07

(52) U.S. Cl. CPC A61B 1/07
USPC 600/178; 600/182

(57) ABSTRACT

Couplings designed and configured to optically couple light conductors in light-conducting cables to tools that require light at working regions of the tools. Examples of such tools include endoscopes and microscopes. Each coupling couples one or more pairs of light conductors, for example, optical fibers, with each other by locating the ends of each pair in confronting relation and by holding the light conductors so that their optical axes are substantially coaxial with one another. In this manner, light is efficiently transmitted through the confronting ends to minimize losses across the interface. Each coupling can include one or more pairs of mechanically interengaging alignment structures for ensuring that the light conductors are aligned properly.

This application claims the benefit of priority of U.S. Provisional Patent Application No. 61/658,350 filed on Jun. 11, 2012, and titled “ENDOSCOPES WITH REDUCED OPTICAL FIBERS,” which is incorporated herein by reference in its entirety.

### FIELD OF THE INVENTION

The present invention generally relates to systems for illuminating, viewing, and imaging objects and remote spaces or cavities. More particularly, the present invention is directed to transmission-efficient light couplings and tools and systems utilizing such couplings.

### BACKGROUND

It is desired for medical endoscopes to consume as small a cross-sectional space as possible in order to allow minimally invasive surgery and fast patient recovery. In the current art, radiation from a single illumination source is focused such that as much radiation as possible enters a fiber optic cable that is secured to the illumination source. The fiber optic cable consists of hundreds to thousands of individual optical fibers contained within a protective jacket or sleeve and secured to mechanical couplings at each end. The opposite end of the fiber optic cable is coupled to an endoscope. The radiation then passes from the first fiber optic cable to another bundle of optical fibers contained within the endoscope and then to the object.

### SUMMARY

In one implementation, the present disclosure is directed to an apparatus. The apparatus includes a tool having a working region requiring light, the tool including a first light conductor having a first end and extending to the working region so as to provide the light when the tool is being used; a light-conducting cable containing a second light conductor having a second end; and an optical coupling designed and configured to removably connect the light-conducting cable to the tool so as to hold the second end of the second light conductor in confronting relation to the first end of the at least one first light conductor so that the at least one second light conductor and the first light conductor have corresponding respective optical axes that are substantially aligned with one another at the first and second ends.

In another implementation, the present disclosure is directed to an apparatus. The apparatus includes an endoscope having a working end requiring light, the endoscope including: a first light conductor having a first end and extending to the working end so as to provide the light when the endoscope is being used; and an optical coupling receiver designed and configured to form an optical coupling with a light-conducting cable containing a second light conductor having a second end fixed relative to the light-conducting cable, the optical coupling receiver designed and configured to hold the first end in axial alignment with the second end of the second light conductor when the light-conducting cable is secured to the optical coupling receiver.

In still another implementation, the present disclosure is directed to an apparatus. The apparatus includes a light-conducting cable designed and configured to be engaged with an optical-coupling receiver of a tool having a working region requiring light, the tool including a first light conductor extending from the optical-coupling receiver to the working region, wherein the light conducting cable includes: a second light conductor designed and configured to transmit light from a light source to the first light conductor of the tool when the light-conducting cable is operatively connected to the optical-coupling receiver; wherein the light-conducting cable is designed and configured to engage the optical-coupling receiver so that the second light conductor is in axial alignment with the first light conductor of the tool.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a combination diagrammatic representation of a conventional endoscope system in which a single illumination source is optically coupled through a fiber optic cable to an endoscope;

FIG. 2 is a combination diagrammatic representation of a conventional endoscope system in which a single illumination source is focused onto one end of a fiber optic cable;

FIG. 3 is a combination diagrammatic representation of a conventional endoscope system in which a fiber optic cable is coupled to an endoscope;

FIG. 4 is a combination diagrammatic representation of an endoscope system made in accordance with the present invention, showing the system as having a single channel for radiation transmission through a series of aligned light conductors;

FIG. 5 is a combination diagrammatic representation of an endoscope system made in accordance with the present invention, showing the system as having multiple channels for radiation transmission through multiple series of aligned light conductors, wherein the multiple channels are contained within a single bundle using connectors that maintain alignment of the individual channels;

FIG. 6A is a diagrammatic representation of an endoscope system made in accordance with the present invention, showing the system as having a single light source and multiple channels for radiation transmission through multiple optically parallel channels, wherein the multiple channels are coupled individually using connectors that maintain alignment of light conductors within the individual channels;

FIG. 6B is a diagrammatic representation of an endoscope system made in accordance with the present invention, showing the system as having multiple light sources and multiple channels for radiation transmission through multiple optically parallel channels, wherein the multiple channels are coupled individually using connectors that maintain alignment of light conductors within the individual channels;

FIG. 7A is a diagrammatic representation of a traditionally sized endoscope having an optical coupling made in accordance with the present invention;

FIG. 7B is a diagrammatic representation of a miniature endoscope made in accordance with the present invention;

FIG. 8 is a diagram illustrating a scheme for manufacturing wafer-based multi-light-conductor positioning structures that can be used to precisely position optical fibers in optical couplings made in accordance with the present invention;

FIG. 9 is a perspective view of a wafer-based alignment structure containing an array of twelve light-conductor-receiving apertures, showing two light conductors engaged with corresponding respective light-conductor-receiving apertures;

FIG. 10 is perspective view illustrating a pair of wafer-based alignment structures, showing how the structures can be configured to confront and engage one another so that corresponding light conductors align with one another;

FIG. 11 is an enlarged perspective view of two pairs of light conductors aligned with one another within an optical coupling of the present disclosure; and

FIG. 12 is an enlarged cross-sectional view of a pair of light conductors aligned with one another within an optical coupling of the present disclosure.

### DETAILED DESCRIPTION

Illumination sources for endoscopes typically include mercury lamps, tungsten halogen bulbs, light emitting diodes (LEDs), and xenon lamps. These sources are not easily focused to small spot sizes for light collection by fiber optic cables. Consequently, the fiber optic cables remain much larger than desired in order to capture sufficient illumination. In an effort to overcome poor collection efficiency between the illumination source and the fiber optic cable, the power of the illumination source is often increased to compensate. This generates additional heat and wasted energy. Additionally, due to the packing characteristics of the fibers within the bundle, the fiber optic cable includes areas of dead space that contain no optical fibers for radiation transmission. As much as thirty percent of what little radiation is made available for the fiber optic cable can be lost.

The connection to the endoscope experiences a similar loss of illumination as a result of dead spaces within the fiber bundle contained within the endoscope. Additional losses occur at the coupling between the fiber optic cable and the endoscope since the fibers within the fiber optic cable and the fibers within the endoscope are not precisely aligned to each other. Losses throughout the system can exceed eighty-three percent of the available radiation. The optical losses represent a significant amount of photonic energy that can cause heating and damage to the endoscopic system unless properly managed, thereby increasing complexity and cost. Furthermore, the significant loss of radiation drives the addition of more optical fibers to compensate for low intensity. The additional optical fibers add complexity, cost, and physical size to the conventional devices.

The present inventor has recognized these issues and has identified that a need exists to devise an efficient coupling of radiation from an illumination source through a fiber optic cable to the object/region such that a smaller fiber optic cable and/or a lower output power source, such as an LED, can be used effectively. It is, accordingly, an aim of the present invention to overcome many of the shortcomings of prior art endoscopic systems and to provide an improved optical illuminating, viewing, and/or imaging system that is uniquely adapted for incorporation in microscopes, endoscopes, and similar devices.

The present invention addresses the problems identified above by providing a novel solution utilizing a single or multitude of individual light conductor(s), such as optical fibers, that are aligned to an illumination source and whose alignment is maintained across boundaries from the illumination source to the object to be illuminated. Furthermore, each individual light conductor within the light-conductor cable may be coupled with a unique illumination source allowing the tailoring of the illumination for useful purposes. It is an important feature of some embodiments that the present invention provides an apparatus and a technique whereby light of varying wavelengths and intensities may be produced by selection of appropriate illumination sources and light conductor(s), such as optical fiber(s), for viewing, analytical purposes, and actual work.

More particularly, embodiments of the present invention are composed of a single or multiple light conductor(s) whose alignment is maintained from the radiation source to the object location. The alignment of the conductors across a continuity break, such as at a connection point, is maintained by mechanical means within the couplings between the light source and the object. Various embodiments find utility as an illumination and imaging source for microscopes, especially for fluorescent imaging and analysis. Other embodiments find use in fiberscopes and medical endoscopes used to view and/or analyze tissues in inaccessible spaces and body cavities.

The present inventions now will be described more fully hereinafter with reference to the accompanying drawings, which illustrate some examples of embodiments and features of the present invention. The use of these examples by no means limits the scope of the invention, as those skilled in the art will recognize the value obtained from various combinations of elements and features of the present invention.

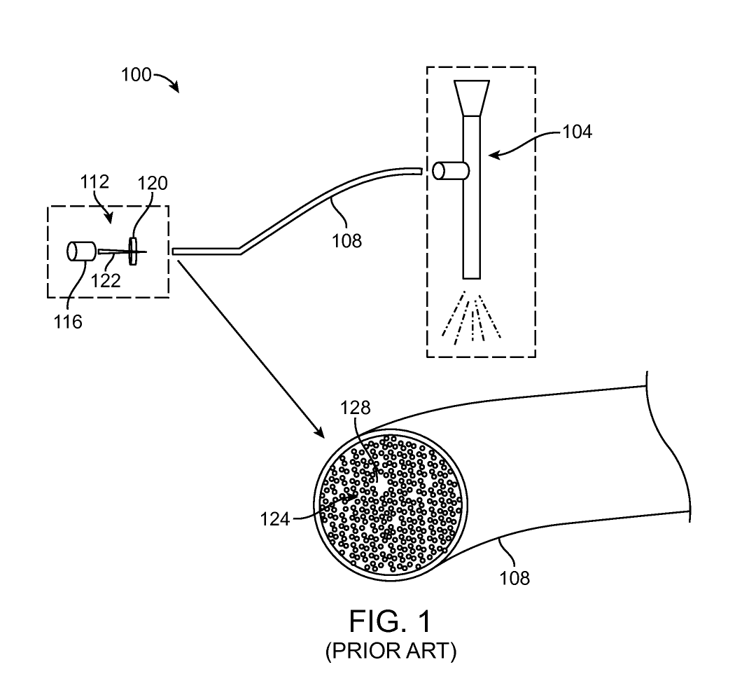

Referring more particularly to the drawings, FIG. 1 depicts a conventional endoscope system 100 that includes an endoscope 104 and a fiber optic cable 108, which are connected together to pass light generated by a light source 112. Light source 112 consists of a lamp 116 and optics 120, which are used to collect light 122 from the lamp and direct it to cable 108. Fiber optic cable 108 comprises hundreds to thousands of randomly positioned individual optical fibers 124 packed together within the cable. Optical fibers 124 are designed to transmit radiation that falls upon the face of the core of each fiber. As those skilled in the art will readily understand, the core of an optical fiber represents a fraction of the cross-sectional area of that fiber. The area surrounding the core is known to those in the art as cladding. Light that enters the cladding is not transmitted through the optical fiber. Within the packing of optical fibers 124 within cable 108, spaces 128 occur between adjacent ones of the fibers that also cannot transmit the light that falls upon them.

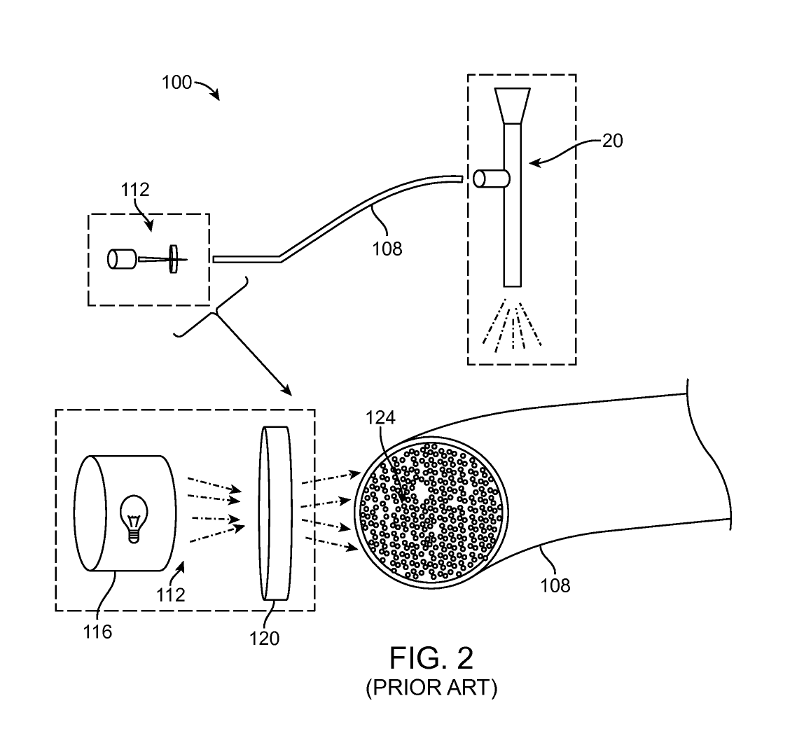

FIG. 2 depicts another view of conventional endoscope system 100 of FIG. 1. In the case of FIG. 2, light from a light source 112 is focused onto fiber optic cable 108, resulting in a decreased percentage of the available light capable of being transmitted through optical fibers 124 with the cable, typically seventy to eighty percent. The lost radiation is mostly absorbed by fiber optic cable 108 and converted to heat, but a fraction of it can be reflected in various directions or returned to light source 112.

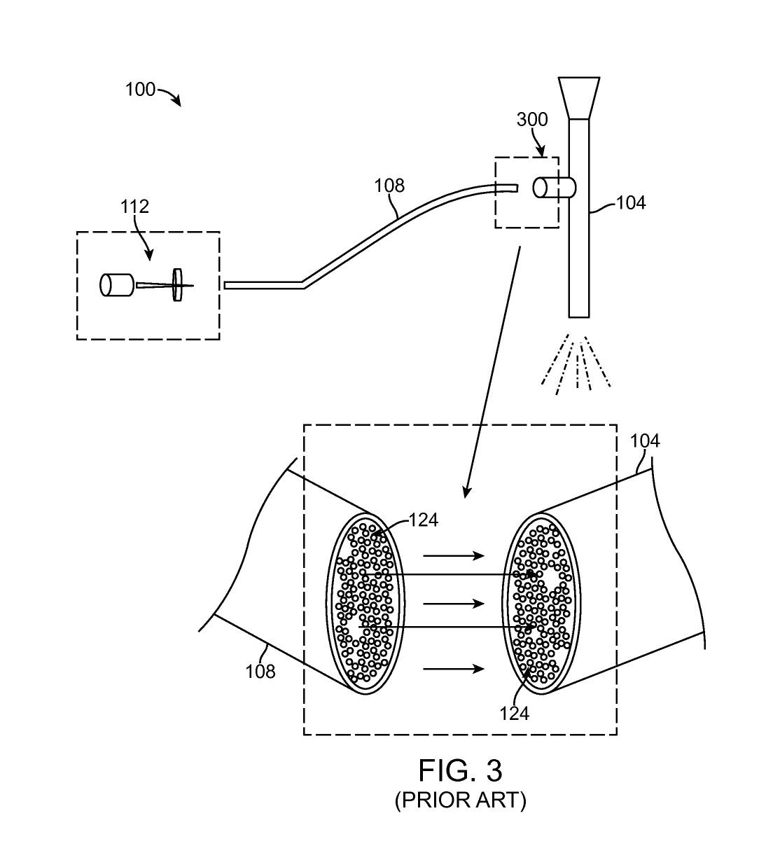

FIG. 3 depicts yet another view of conventional endoscope system 100 of FIG. 1. FIG. 3 shows the details regarding the connection 300 of fiber optic cable 108 to endoscope 104. Both endoscope 104 and fiber optic cable 108 contain numerous individual optical fibers 124 randomly arranged within their connector assemblies (not shown), but the randomness is illustrated in the enlarged detail view of FIG. 3. Light entering endoscope 104 suffers from the same losses as the light that entered fiber optic cable 108 from light source 112, resulting in an additional twenty to thirty percent loss and more heating of the components. Further, additional light is lost since individual fibers 124 within the cable 108 and endoscope 104 are not aligned to each other. The total losses throughout endoscope system 100 can approach eighty-three percent.

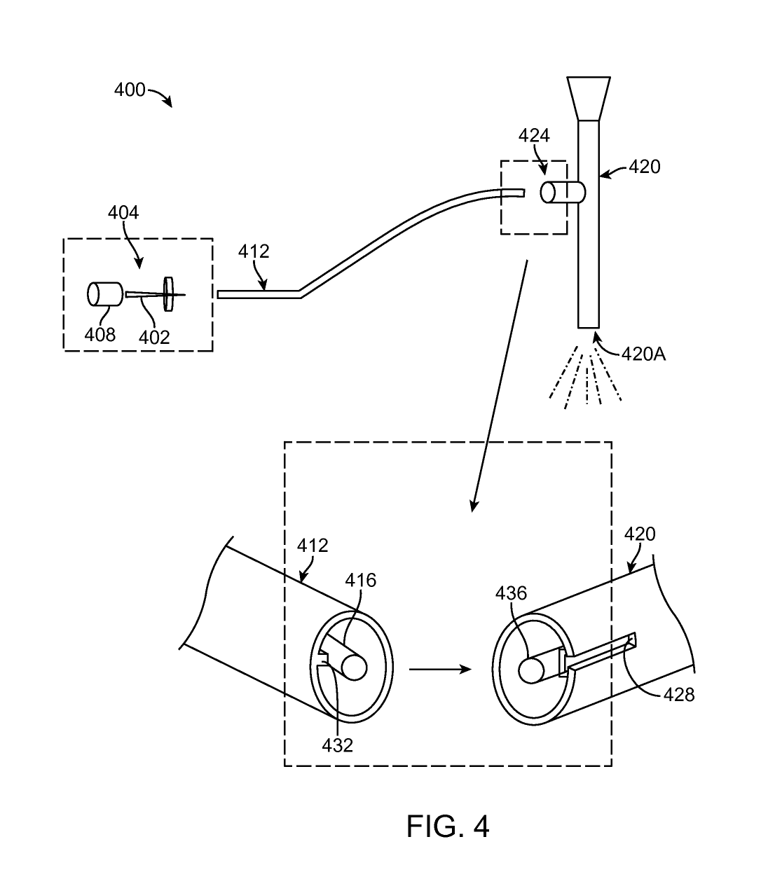

FIG. 4 depicts an exemplary system 400 made in accordance with the present invention. In the embodiment illustrated, the output 402 from a light source 404 (here, having a single light-emitting element 408, such as an LED or laser diode) is focused onto a light-conducting cable 412, which in this example includes a single light conductor, here, a single optical fiber 416. Cable 412 connects to a tool 420 via a coupling 424. In this example, tool 420 comprises an endoscope. However, in other embodiments, tool 420 can be another tool having a working end 420A that requires light from one or more light sources, such as light source 404. Examples of tools that can be used as tool 420 include, but are not limited to microscopes, fiber optic inspection systems, video scopes, semiconductor inspection equipment, jet engine inspection tools and endoscopes, among others. Coupling 424 utilizes mechanical means, such as a groove 428 and matching spline 432 interengaging arrangement shown, to create and maintain alignment between fiber 416 in cable 412 and a corresponding single fiber 436 of tool 420. Other examples of mechanical means that can be used are a pin and slot arrangement and a key and keyway arrangement, including arrangements having multiple ones of each of these arrangements and combinations of these arrangements, among many others. Those skilled in the art will readily appreciate the wide variety of mechanical means and mating-part arrangements that can be used to create and maintain alignment between fibers 416 and 436. In addition, because of the wide variety and ubiquity of such mechanical means, skilled artisans will also understand that any lack of exhaustive listing of suitable mechanical means does not prevent them from practicing the present invention to its fullest scope.

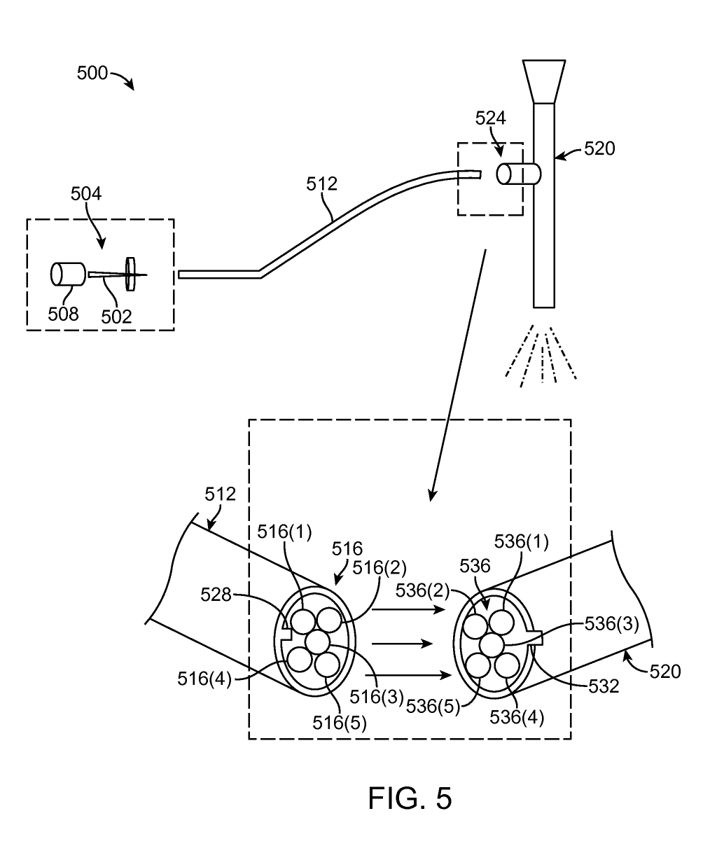

FIG. 5 depicts an exemplary endoscope system 500 made in accordance with the present invention. In this embodiment, the output 502 from a single light source 504 (which here is depicted as including a single light-emitting element 508 like element 408 of FIG. 4 but that could include multiple light-emitting elements) is focused onto a light-conducting cable 512 comprising a plurality of light conductors 516, here five optic fibers 516(1) to 516(5) arranged in nonrandom positions so as to form a predetermined fixed arrangement. Cable 512 connects to an endoscope 520 via a coupling 524 that uses mechanical means, here, pin 528 and slot 532, to create and maintain the alignment between fibers 516(1) to 516(5) in cable 512 and matching the predetermined fixed arrangement of optical fibers 536(1) to 536(5) in the endoscope. As those skilled in the art will readily appreciate, the mechanical means for aligning fibers 516(1) to 516(5) with corresponding respective fibers 536(1) to 536(5) can be any of a wide variety of mechanical means, such as means that are the same as or similar to the mechanical mean noted above relative to FIG. 4. As those skilled in the art will readily appreciate, endoscope 520 can be replaced with another tool, such as a microscope, surgical headlamp, or flexible video scope, among others.

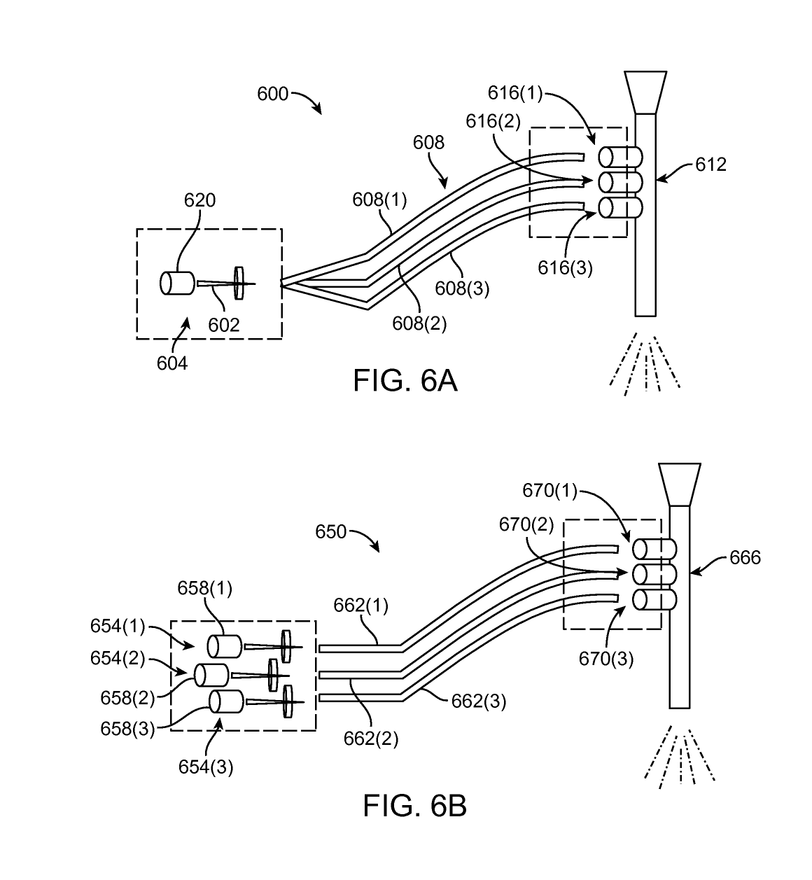

FIGS. 6A and 6B depict, respectively, yet other exemplary endoscope systems 600 and 650 made in accordance with the present invention. In FIG. 6A, the output 602 from a single light source 604 is focused onto a plurality of light cables 608, here cables 608(1) to 608(3), closely packed together near the light source to receive the focused output. Optical cables 608(1) to 608(3) are optically connected to an endoscope 612 using corresponding individual couplings 616(1) to 616(3), which mechanically maintain alignment of matching individual light conductors (not shown) of cables 608(1) to 608(3) and couplings 616(1) to 616(3), for example, in the same manner depicted in the enlarged details of FIG. 4. Alternatively, one or more of cables 608 can each be replaced with multi-conductor light-conducting cables, such as cable 512 illustrated in endoscope system 500 of FIG. 5.

In the present example of FIG. 6A, endoscope system 600 can be said to have three channels (corresponding to three light-conducting cables 608). It is noted that in some embodiments, the character of the light in each of light-conducting cables 608 can be the same as the character of the light in one or both of the other cables if the light conductors (not shown) of the cables are identical in optical properties. However, in other embodiments the character of the light can differ among light-conducting cables 608 in one or more desired ways by appropriate choice of the optical properties of the individual light conductors within the cables. Those skilled in the art will understand how to tune the individual light conductors within the cables 608 to suit the desired application.

In addition, although a single light source 604 (here having a single light-emitting element 620) is illustrated, the single light source can be replaced with multiple light sources, for example, with the multiple light outputs being directed into the multiple light-conducting cables 608 in essentially the manner shown in endoscope system 650 of FIG. 6B or in multiple combined groupings in the manner of the combined grouping illustrated in endoscope system 600 of FIG. 6A. Additional optics may be needed to focus the light from multiple sources into each of the one or more combined fiber groupings, and those skilled in the art will understand how to focus the light from the multiple sources to accomplish the desired goals. The multiple light sources can be different from one another in any of a variety of ways, such as differing emissions spectra, differing types (e.g., LED, laser diode, xenon arc, etc.), differing powers, etc.

In FIG. 6B, endoscope system 650 includes multiple light sources 654(1) to 654(3) (here, each having a single radiation-emitting element 658(1) to 658(3)) that are focused onto corresponding respective individual light-conducting cables 662(1) to 662(3). Light-conducting cables 662(1) to 662(3) are connected to an endoscope 666 using corresponding individual couplings 670(1) to 670(3) that mechanically maintain alignment of matching individual light conductors (not shown) of cables 662(1) to 662(3) and couplings 670(1) to 670(3). Each of light-conducting cables 662(1) to 662(3) may comprise a single light conductor, such as seen in cable 412 in the enlarged detail of FIG. 4, or a plurality of light conductors bundled together, such as shown in cable 512 in the enlarged detail of FIG. 5. In the same manner as described above, individual ones of multiple light sources 654(1) to 654(3) can be of any suitable type and/or can be replaced with multiple light sources, and the light conductors of the light-conducting cables 662(1) to 662(3) can be tuned in any manner desired to suit the corresponding respective one(s) of the light sources.

It is well known that a single light source such as xenon, metal halide, halogen, tungsten bulb, LED, etc., suffers limitations, such as etendue, that restrict the ability to concentrate the radiation to a small spot. For example, while LEDs have desirable properties as an illumination source, unfortunately they lack the concentrated intensity that would permit them to be focused onto small light conductors, for example, optical fibers. However, in one embodiment of the present invention individual LEDs are coupled to either individual light conductors (e.g., optical fibers) or small clusters of individual light conductors (e.g., optical fibers), thereby allowing increased amounts of radiation at the distal end of the fiber. Furthermore, the use of multiple sources, in this example LEDs, allows different sources to be selected for different purposes. A combination of visible, ultraviolet, and infrared sources allows the visible radiation to be used for purposes of general illumination, while the ultraviolet radiation could be controlled separately for fluorescence imaging while the infrared radiation can be used for tissue stimulation or phototherapy. U.S. patent application Ser. No. 13/486,082 titled “Multi-Wavelength Multi-Lamp Radiation Sources and Systems and Apparatuses Incorporating Same” of Cogger et al. (“the '082 application”) discloses unique arrangements and combinations of radiation sources, as well as radiation combiners that can be used to combine the various forms of radiation generated by those sources. The '082 is incorporated herein by reference for all of its disclosure on these topics. As those skilled in the art will readily appreciate, the radiation sources, radiation combiners, and other embodiments and features disclosed in the '082 application can be used in place of the light sources disclosed in this current disclosure.

In a specific example, the output of the light source 404 of FIG. 4 can be efficiently coupled with endoscope 420 using a single optical fiber 416 of 0.5 millimeter diameter core or 0.2 square millimeter delivering 300 lumens of light to the object through the endoscope when the boundary connections at the coupling 424 are mechanically constrained in position and orientation. In comparison, a fiber optic bundle of 6.55 square millimeters with randomly oriented fibers at connection boundaries can deliver only 160 lumens under similar conditions.

In another embodiment, the output of the light sources 654(1) to 654(3) of FIG. 6B can be coupled with a microscope system (not shown, but in lieu of endoscope 666 using liquid light guides or optical fibers. The light sources 654(1) to 543(3) of FIG. 6B can comprise a combination of laser, LED, metal halide, xenon or other sources. The use of different sources allows the tailoring of the radiation based on the unique property of each source. For example, a configuration of light sources 654(1) to 654(3) can comprise three lasers of red, green, and blue. Each of the lasers may be individually adjustable in its intensity, while the red laser and the green laser are split into two outputs each of the type shown in FIG. 6A. Each of the five outputs is directed to a microscope consisting of an imaging and detection system for the excitation and detection of a fluorophore. Although the system consists of only three lasers, the microscope can be configured to detect five types of fluorophores. The '082 application mentioned above discloses a number of embodiments using red, green, and blue lasers, and those embodiments are incorporated herein by reference for all they teach that can be incorporated into a red, green, and blue light based system that incorporates one or more of the features disclosed in the current application.

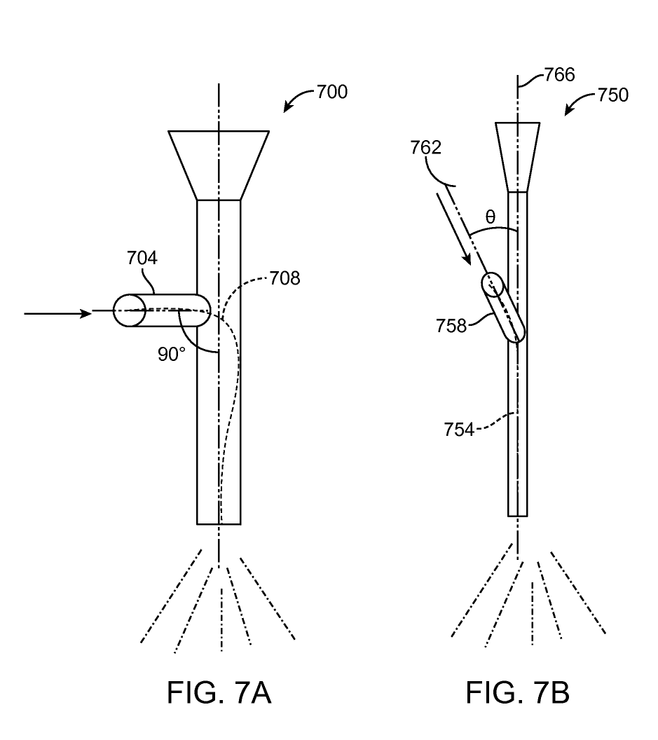

FIG. 7A shows a traditionally sized endoscope 700 that includes an optical coupling receiver 704 of the present invention in which an optical fiber 708 transitions 90° from the coupling receiver to the interior of the endoscope. As is customary with endoscopes of this size, optical fiber 708 can be gently wrapped around a central optical element (not shown) that is typically provided for viewing. Optical coupling receiver 704 can be adapted to be part of an optical couple of the present invention, such as an optical coupling that is the same as or similar to single-fiber optical coupling 424 of FIG. 4. In alternative embodiments, optical coupling 704 can be replaced with a multi-fiber optical coupling, such as multi-fiber optical coupling 524 of FIG. 5. In addition, endoscope 700 of FIG. 7A can readily be modified to include multiple optical couplings in the manner of the embodiments shown in FIGS. 6A and 6B.

FIG. 7B shows a unique endoscope 750 that is so small relative to the diameter of the light conductor 754 that the light conductor cannot handle a 90° transition from a conventionally oriented 90° coupling receiver (not shown, but similar to coupling receiver 704 of FIG. 7A) to the interior of the endoscope. In the example of FIG. 7B, endoscope 750 includes a coupling receiver 758 attached to the endoscope so that the angle ⊖ between the longitudinal axis 762 of the coupling receiver relative to the longitudinal axis 766 of the endoscope above the intersection of the coupling with the endoscope is less than 90°. In the example shown, angle ⊖ is 45°, but it could be more or less. As those skilled in the art will readily appreciate, providing an acute angle like this results in a larger radius transition in light conductor 754, which ultimately allows endoscope 750 to be made very small while retaining the superior transmission properties that the specially configured alignment that coupling receiver 758 affords, allowing such small endoscopes to provide high intensity and high quality illumination. As those skilled in the art will readily understand, coupling receiver 758 can be designed and configured for any suitable coupling, which may be the same as or similar to either of the couplings 424 and 524 of FIGS. 4 and 5, respectively, depending on how many fibers are present. In addition, those skilled in the art will also readily appreciate that an endoscope utilizing an acute-angle coupling arrangement, such as the arrangement shown in FIG. 7B, can be used in a multi-channel, multi-coupling embodiment in a manner similar to the embodiments shown in FIGS. 6A and 6B.

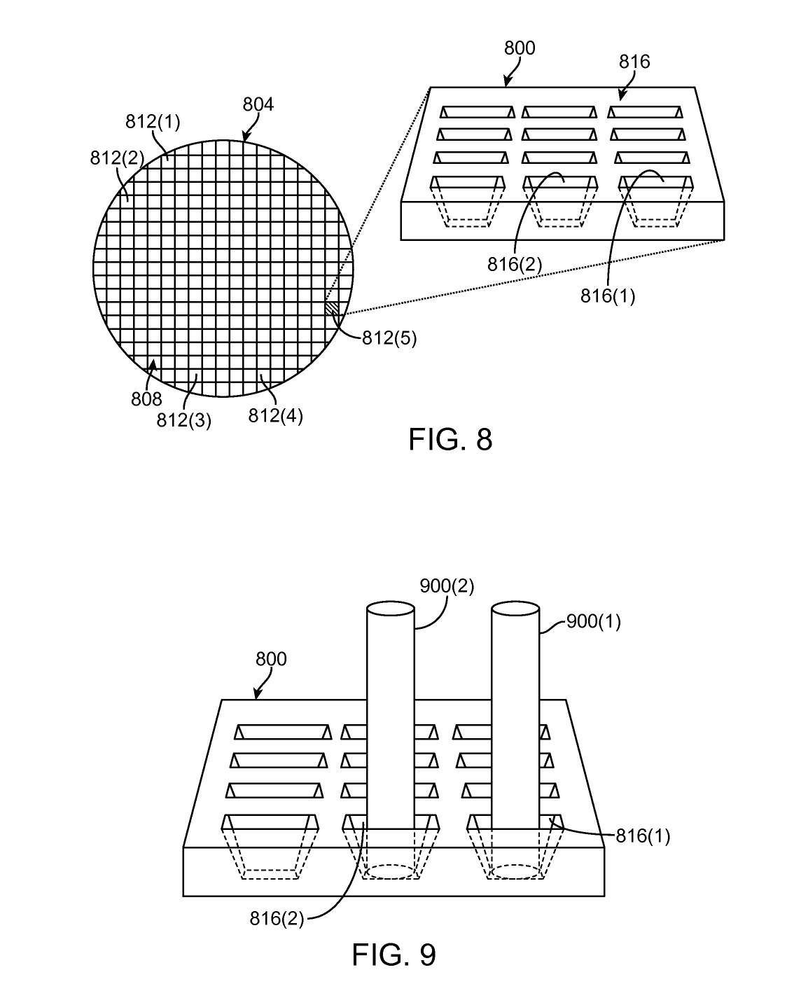

FIG. 8 illustrates a scheme for creating light-conductor-positioning structures (one structure 800 is shown enlarged in FIG. 8) from a wafer 804, such as a silicon wafer or wafer of another material or materials, as a starting point. In this example, wafer 804 is processed, for example, using conventional microelectronics wafer-processing techniques, such as lithography and etching techniques. As illustrated by the grid pattern 808 on wafer 804 that defines individual dice 812 (only a few dice 812(1) to 812(5) individually labeled for convenience), the wafer can be used to create multiple light-conductor-positioning structures that are the same as or similar to the light-conductor-positioning structure 800 shown, with each such light-conductor-positioning structures corresponding to a respective one of the dice. In the example shown, light-conductor-positioning structure 800 has an array of twelve light-conductor-positioning apertures 816, two of which, i.e., apertures 816(1) and 816(2) (the rest are not individually labeled for convenience) are engaged by a pair of corresponding light conductors 900(1) and 900(2) in FIG. 9. Of course, having twelve positioning apertures 816, positioning structure 800 can accommodate up to twelve light conductors with one conductor in each aperture. Those skilled in the art will readily appreciate that light-conductor-positioning structures in accordance with the present invention can be any suitable size, including micro-size. It is noted that while light-conductor-positioning structure 800 is shown as being rectangular, it can be any other shape desired, such as circular, among others. Likewise, the light-conductor-positioning apertures can be arranged in any desired pattern, such as a ring pattern, among others.

Referring to FIG. 9, each fiber-alignment aperture 816 can be tapered, for example, to assist in the engagement of the fibers 900(1) and 900(2) with the apertures. This tapering can be created in any suitable manner. For example, the taper can simply be the artifact of conventional wet-etching techniques that can be used to create the apertures. Techniques for processing wafers are well known in the art, such that they need not be described in any detail herein for those skilled in the art to understand how fiber-positioning structures of the present invention can be made. That said, apertured fiber-positioning structures can be made using any suitable techniques, such as chemical and other types of etching, laser ablation, mechanical milling, high-pressure-fluid milling, electrical milling, and additive manufacturing, among others.

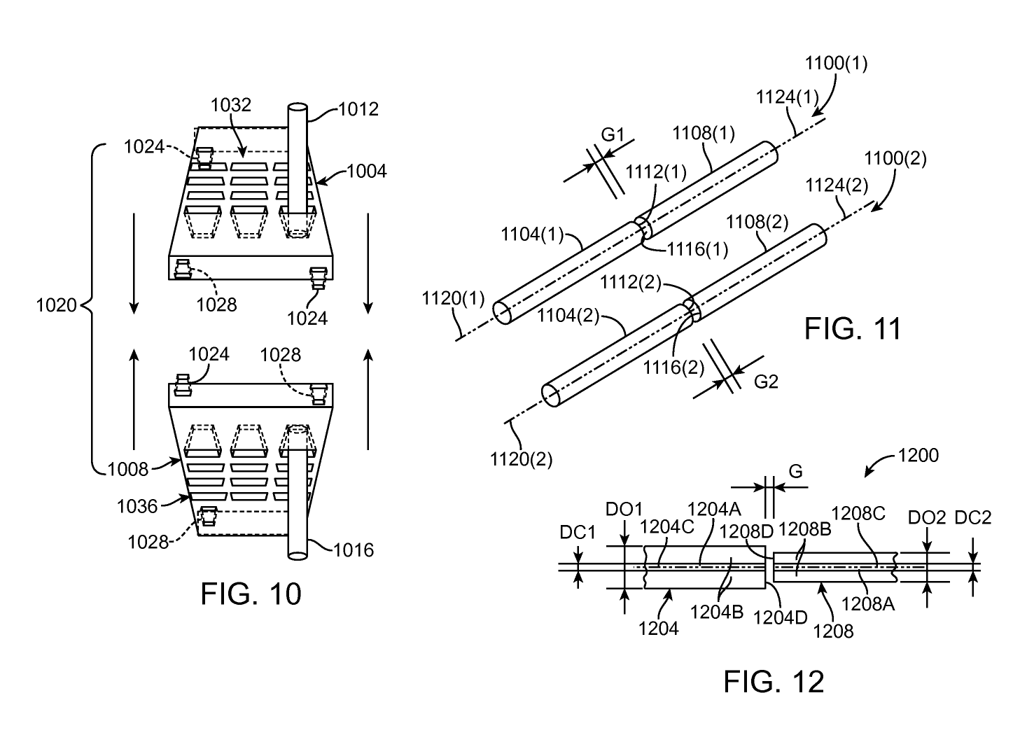

FIG. 10 illustrates how a pair of like light-conductor-positioning structures 1004 and 1008 are used to align the ends of one or more pairs of light conductors, here light conductors 1012 and 1016, with one another in a coupling 1020, which can be similar to coupling 424 of FIG. 4 or coupling 524 of FIG. 5, among others. To facilitate the alignment of light-conductor-positioning structures 1004 and 1008 with one another, each of them includes one or more alignment features, here pins 1024 and corresponding receivers 1028. When the corresponding pin 1024/pin receiver 1028 pairs are fully engaged with one another, coupling 1020 is complete, i.e., the apertures 1032 and 1036 in the respective light-conductor-positioning structures 1004 and 1008 are in registration with one another and any light conductors, here light conductors 1012 and 1016, in opposing apertures are fully aligned with one another with respect to their optical axes. As those skilled in the art will readily appreciate, pins 1024 and pin-receivers 1028 can be made in any suitable manner, including using any one or more of the techniques noted above. Of course, the alignment features can be any suitable features other than pins and pin-receivers, such as the alignment means noted above relative to FIGS. 4 and 5.

FIG. 11 illustrates two pairs 1100(1) and 1100(2) of light conductors 1104(1) and 1104(2) and 1108(1) and 1108(2) that are aligned with one another as they would be inside an optical coupling (not shown) of the present disclosure, such as within multi-light-conductor optical coupling 524 of FIG. 5. As can be readily understood, light conductors 1104(1) and 1104(2) can be part of a light-conducting cable (not shown, but it can be similar to any of light cables 412, 512, 608, and 662 of FIGS. 4, 5, 6A, and 6B, respectively), and light conductors 1108(1) and 1108(2) can be part of a tool or device, such as an endoscope (not shown, but it can be similar to any of endoscopes 420, 520, 612, 666 of FIGS. 4, 5, 6A, and 6B, respectively). As seen in FIG. 11, when the mechanically interengaging elements (not shown) of the optical coupling are fully engaged with one another, the ends 1112(1) and 1116(1) of light conductors 1104(1) and 1108(1), respectively, and the ends 1112(2) and 1116(2) of light conductors 1104(2) and 1108(2), respectively, confront one another so that they form gaps G1 and G2, respectively. Gaps G1 and G2 can range anywhere from zero mm to 2 mm or more, depending on design parameters. The most desirable gap is closest to zero, however, a suitable gap, meaning one which can still deliver most of the radiation across its distance, has an upper limit related to the numerical aperture of the sending light conductor, the numerical aperture (NA) of the receiving light conductor and the nature of the space between them (e.g., reflective tubing, or air gap or something else). In the ideal case, the NA of the receiving fiber would be greater than the sending fiber. As can be readily appreciated, when the optical coupling is properly made, the optical axes 1120(1) and 1124(1) of light conductors 1104(1) and 1108(1), respectively, are substantially coaxial at the confrontation of ends 1112(1) and 1116(1), as are optical axes 1120(2) and 1124(2) at ends 1112(2) and 1116(2) of light conductors 1104(2) and 1108(2).

FIG. 12 illustrates a pair 1200 of light conductors 1204 and 1208 that are also aligned with one another as they would be inside an optical coupling (not shown) of the present disclosure, such as any of optical couplings 424, 524, 616, and 670 of FIGS. 4, 5, 6A, and 6B, respectively. In FIG. 12, each light conductor 1204 and 1208 includes a light-conducting core 1204A and 1208A and a corresponding cladding 1204B and 1208B, as is well known in the art. Each core 1204A and 1208A has a corresponding optical axis 1204C and 1208C and an effective diameter DC1 and DC2, and each light conductor 1204 and 1208 has an overall diameter DO1 and D02. If the transverse cross-sectional shapes of cores 1204A and 1208A and/or the overall transverse cross-sectional shapes of light conductors 1204 and 1208 are not circular, effective diameter DC1 and DC2 and/or overall diameters DO1 and DO2 can be another, appropriate dimension, such as a maximum “diameter,” average “diameter,” etc. In some examples, such as when light conductors 1204 and 1208 are optical fibers having cores and overall fibers that have circular transverse cross-sectional shapes, core diameters DC1 and DC2 may range from 7 microns to 1000 microns and overall diameters DO1 and DO2 may range from 30 microns to 1035 microns. It is noted that these dimensions are not necessarily limiting but are representative in the context of typical endoscopy applications.

It is noted that core diameters DC1 and DC2 need not necessarily be the same as one another. Similarly, overall diameters DO1 and DO2 need not be the same as one another. Generally, a goal of the alignment means described herein is to ensure that the optical axes of the confronting light conductors, such as optical axes 1204C and 1208C of light conductors 1204 and 1208, respectively, coincide as closely as practicable at confronting ends 1204D and 1208D so that the maximum amount of light is passed from one light conductor to the other through the confronting ends. As those skilled in the art will readily appreciate, the amount of error in the coincidence of the optical axes of two confronting light conductors that is tolerable will vary depending on one or more factors, such as the sizes of the light conductors and the relative transverse cross-sectional areas of the light-conducting portions of the conductors and the direction of light conduction, if the light-conducting portions have identical transverse cross-sectional areas having diameters in a range of 50 micron to 500 micron, then it is desirable that the error in the alignment of the optical axes be less than about 10% of the diameter. Misalignment is best specified as a percentage versus an absolute number since its impact on transmission across the boundary is proportional to the overlapping area.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. An apparatus, comprising:
a tool having a working region requiring light, said tool including a first light conductor having a first end and extending to said working region so as to provide the light when the tool is being used;
a light-conducting cable containing a second light conductor having a second end; and
an optical coupling designed and configured to removably connect said light-conducting cable to said tool so as to hold said second end of said second light conductor in confronting relation to said first end of said at least one first light conductor so that said at least one second light conductor and said first light conductor have corresponding respective optical axes that are substantially aligned with one another at said first and second ends.

2. An apparatus according to claim 1, wherein said tool is an endoscope.

3. An apparatus according to claim 1, further comprising a light source designed and configured to provide the light, said light source being optically located to input light into said light-conducting cable at an end of said light-conducting cable optically opposite said optical coupling.

4. An apparatus according to claim 1, wherein said first and second optical conductors are optic fibers each having a core and optical cladding.

5. An apparatus according to claim 1, wherein said optical coupling includes interengaging mechanical elements on said light-conducting cable and said tool that are designed and configured to ensure that first and second ends of said first and second light conductors, respectively, are aligned with one another when said optical coupling is fully made.

6. An apparatus according to claim 1, wherein:
said tool includes a plurality of first light conductors extending from said optical coupling to said working region;
said light-conducting cable includes a plurality of second light conductors at said optical coupling; and
said optical coupling is designed and configured to hold each of said plurality of second light conductors in fixed relation to said plurality of first light conductors so that ends of said plurality of second light conductors confront corresponding respective ends of said plurality of first light conductors and optical axes of said plurality of second light conductors are substantially coincidental with corresponding respective optical axes of said plurality of first light conductors at said ends.

7. An apparatus according to claim 6, wherein said optical coupling includes interengaging mechanical elements on said light-conducting cable and said tool that are designed and configured to ensure that said optical axes of said plurality of first light conductors are substantially aligned with said optical axes of said plurality of second light conductors when said optical coupling is fully made.

8. An apparatus according to claim 6, further comprising a single light source designed and configured to simultaneously provide light to all of said plurality of second light conductors, said light source being optically located to input light into said plurality of second light conductors at an end of said light-conducting cable optically opposite said optical coupling.

9. An apparatus according to claim 6, further comprising a plurality of light sources designed and configured to provide light to individual ones of said plurality of second light conductors, said plurality of light sources being optically located to input light into corresponding respective ones of said plurality of second light conductors at ends of said plurality of second light conductors optically opposite said optical coupling.

10. An apparatus according to claim 6, wherein said tool comprises an endoscope.

11. An apparatus according to claim 1, further comprising a plurality of optical couplings, wherein said tool includes a plurality of first light conductors extending from corresponding respective ones of said plurality of optical couplings to said working region.

12. An apparatus according to claim 11, further comprising a plurality of light-conducting cables containing, correspondingly, a plurality of second light conductors, wherein each of said plurality of optical couplings is designed and configured to removably connect that one of said plurality of light-conducting cables to said tool so as to hold an end of a corresponding one of said plurality of second light conductors in aligned confronting relation to an end of a corresponding one of said plurality of first light conductors.

13. An apparatus according to claim 12, wherein each of said plurality of optical couplings includes interengaging mechanical elements on the corresponding one of said plurality of light-conducting cables and said tool that are designed and configured to ensure that confronting ends of corresponding respective ones of said pluralities of first and second light conductors are aligned with one another when said optical coupling is fully made.

14. An apparatus according to claim 12, further comprising a plurality of light sources providing light, correspondingly, to said plurality of second light conductors.

15. An apparatus according to claim 1, wherein said tool comprises:
an endoscope having a working end and a first longitudinal axis; and
an optical coupling receiver fixedly secured to said endoscope and having a second longitudinal axis, wherein said optical coupling receiver forms part of said coupling;
wherein:
said first and second longitudinal axes form a first angle with one another that is less than 90° with said second longitudinal axis being canted away from said working end; and
said first light conduit extends through said optical coupling receiver and to said working end of said endoscope so as to form a second angle greater than 90°.

16. An apparatus according to claim 15, wherein said first angle is about 45°.

17. An apparatus according to claim 1, wherein said coupling includes a first light-conductor-positioning structure secured to said tool and a second light-conductor-positioning structure secured to said light-conducting cable.

18. An apparatus according to claim 17, wherein said first light-conductor-positioning structure includes a first preformed light-conductor-positioning aperture receiving said first light conductor, and said second light-conductor-positioning structure includes a second preformed light-conductor-positioning aperture receiving said second light conductor.

19. An apparatus according to claim 18, wherein said first and second light-conductor-positioning structures include interengaging alignment features designed and configured to engage one another when said coupling is made.

20. An apparatus according to claim 18, wherein each of said first and second preformed light-conductor-positioning apertures is tapered to facilitation installation, respectively, of said first and second light conduits.

21. An apparatus, comprising:
an endoscope having a working end requiring light, said endoscope including:
a first light conductor having a first end and extending to said working end so as to provide the light when the endoscope is being used; and
an optical coupling receiver designed and configured to form an optical coupling with a light-conducting cable containing a second light conductor having a second end fixed relative to the light-conducting cable, said optical coupling receiver designed and configured to hold said first end in axial alignment with the second end of the second light conductor when the light-conducting cable is secured to said optical coupling receiver.

22. An apparatus according to claim 21, wherein said optical coupling includes a first alignment structure designed and configured to engage a second alignment structure on the light-conducting cable when the light-conducting cable is fully secure to said optical coupling receiver, wherein said first and second alignment structures are designed and configured to ensure that the light-conducting cable is engaged with said optical coupling receiver so as to effect the axial alignment between said first end of said first light conductor and the second end of the second light conductor.

23. An apparatus according to claim 21, wherein said endoscope includes a first light-conductor-positioning structure having a first preformed light-conductor-positioning aperture receiving said first light conductor.

24. An apparatus according to claim 23, wherein the light-conducting cable includes a second light-conductor-positioning structure that includes a first alignment structure and a second preformed light-conductor-positioning aperture receiving the second light conductor, said first light-conductor-positioning structure including a second alignment structure designed and configured to engage the first alignment structure so as to axially align the second light conductor with said first light conductor.

25. An apparatus according to claim 21, wherein:
said endoscope has a first longitudinal axis extending through said working end;
said optical coupling receiver has a second longitudinal axis;
said first and second longitudinal axes form a first angle with one another that is less than 90° with said second longitudinal axis being canted away from said working end; and
said first light conduit extends through said optical coupling receiver and to said working end of said endoscope so as to form a second angle greater than 90°.

26. An apparatus according to claim 21, wherein said endoscope includes:
a plurality of first light conductors having corresponding respective first ends and extending to said working end so as to provide the light when the endoscope is being used; and
an optical coupling receiver designed and configured to form an optical coupling with a light-conducting cable containing a plurality of second light conductors having corresponding respective second ends fixed relative to the light-conducting cable, said optical coupling receiver designed and configured to hold each of said first ends in axial alignment with a corresponding one of said second ends of the plurality of second light conductors when the light-conducting cable is secured to said optical coupling receiver.

27. An apparatus according to claim 26, wherein said optical coupling includes a first alignment structure designed and configured to engage a second alignment structure on the light-conducting cable when the light-conducting cable is fully secure to said optical coupling receiver, wherein said first and second alignment structures are designed and configured to ensure that the light-conducting cable is engaged with said optical coupling receiver so as to effect the axial alignment of the second ends of the plurality of second light conductors with corresponding respective said first ends of said plurality of second light conductors.

28. An apparatus according to claim 27, wherein said endoscope includes a first light-conductor-positioning structure having a plurality of first preformed light-conductor-positioning apertures correspondingly respectively receiving said plurality of first light conductors.

29. An apparatus according to claim 28, wherein the light-conducting cable includes a second light-conductor-positioning structure that includes a first alignment structure and a second plurality of preformed light-conductor-positioning apertures correspondingly respectively receiving the plurality of second light conductors, said first light-conductor-positioning structure including a second alignment structure designed and configured to engage the first alignment structure so as to axially align the plurality of second light conductors correspondingly respectively with said plurality of first light conductors.

30. An apparatus according to claim 21, wherein said endoscope includes:
a plurality of first light conductors having corresponding respective first ends and extending to said working end so as to provide the light when the endoscope is being used; and
a plurality of optical coupling receivers each designed and configured to form an optical coupling with a corresponding one of a plurality of light-conducting cables each containing a second light conductor having a second end fixed relative to the light-conducting cable, each of said plurality of optical coupling receivers designed and configured to hold a corresponding respective one of said first ends in axial alignment with a corresponding one of said second ends of the plurality of second light conductors when the plurality of light-conducting cables are secured to said plurality of optical coupling receivers.

31. An apparatus according to claim 30, wherein each of said plurality of optical couplings includes a first alignment structure designed and configured to engage a second alignment structure on a corresponding one of the plurality of light-conducting cables when that one of the plurality of light-conducting cables is fully secure to said optical coupling receiver, wherein said first and second alignment structures are designed and configured to ensure that that one of the plurality of light-conducting cables is engaged with said optical coupling receiver so as to effect the axial alignment between said first end of a corresponding one of said plurality of first light conductors and a corresponding one of the plurality of second ends.

32. An apparatus according to claim 30, wherein said endoscope includes a plurality of first light-conductor-positioning structures each having a first preformed light-conductor-positioning aperture receiving a corresponding one of said plurality of first light conductors.

33. An apparatus according to claim 32, wherein each of the plurality of light-conducting cables includes a second light-conductor-positioning structure that includes a first alignment structure and a second preformed light-conductor-positioning aperture receiving a corresponding one of the plurality of second light conductors, each of said plurality of first light-conductor-positioning structures including a second alignment structure designed and configured to engage a corresponding one of the plurality of first alignment structures so as to axially align a corresponding one of the plurality of second light conductors with a corresponding one of said plurality of first light conductors.

34. An apparatus, comprising:
a light-conducting cable designed and configured to be engaged with an optical-coupling receiver of a tool having a working region requiring light, the tool including a first light conductor extending from the optical-coupling receiver to the working region, wherein
said light conducting cable includes:
a second light conductor designed and configured to transmit light from a light source to the first light conductor of the tool when said light-conducting cable is operatively connected to the optical-coupling receiver;
wherein said light-conducting cable is designed and configured to engage the optical-coupling receiver so that said second light conductor is in axial alignment with the first light conductor of the tool.

35. An apparatus according to claim 34, wherein the optical-coupling receiver includes a first alignment structure designed and configured to engage a second alignment structure on said light-conducting cable when said light-conducting cable is fully secure to the optical coupling receiver, wherein the first alignment structure and said second alignment structure are designed and configured to ensure that said light-conducting cable is engaged with said optical coupling receiver so as to effect the axial alignment between the first light conductor and said second light conductor.

36. An apparatus according to claim 34, wherein said light-conducting cable further includes a first light-conductor-positioning structure that includes a first preformed light-conductor-positioning aperture receiving said second light conductor.

37. An apparatus according to claim 36, wherein the optical-coupling receiver includes a second light-conductor-positioning structure that includes a first alignment structure and a second preformed light-conductor-positioning aperture receiving the first light conductor, said first light-conductor-positioning structure further including a second alignment structure designed and configured to engage the first alignment structure when said light-conducting cable is fully engaged with the optical-coupling receiver.

38. An apparatus according to claim 36, wherein said first preformed light-conductor-positioning aperture is tapered to facilitate engagement of said second light conductor therein.

39. An apparatus according to claim 34, wherein the tool includes a plurality of first light conductors extending from the optical-coupling receiver to the working region, the plurality of first light conductors having a predetermined fixed arrangement relative to one another at the optical-coupling, said light-conducting cable including a plurality of second light conductors and a light-conductor-positioning structure designed and configured to hold said plurality of second light conductors in the predetermined fixed arrangement proximate the optical-coupling receiver when said light-conducting cable is connected to the optical-coupling receiver.

40. An apparatus according to claim 39, wherein said light-conducting cable includes a light-conductor-positioning structure containing a plurality of light-conductor-positioning apertures receiving said plurality of second light conductors so as to hold said plurality of second light conductors in the predetermined fixed arrangement.

41. An apparatus according to claim 40, wherein each of said plurality of light-conductor-positioning apertures is tapered to facilitate engagement of said plurality of second light conductors therein.
